(19) United States
(12) Patent Application Publication
(10) Pub. No.: US 2025/0239367 A1
(43) Pub. Date: Jul. 24, 2025

(54) DEVICE AND METHOD FOR PREDICTING DISEASE OF INTEREST ON BASIS OF DEEP NEURAL NETWORK, AND COMPUTER-READABLE PROGRAM THEREFOR

(71) Applicant: CHUNG ANG UNIVERSITY INDUSTRY ACADEMIC COOPERATION FOUNDATION, Seoul (KR)

(72) Inventor: Yoon-Sik CHO, Seoul (KR)

(21) Appl. No.: 18/852,857

(22) PCT Filed: Mar. 30, 2023
(86) PCT No.: PCT/KR2023/004321

§ 371 Date: Jan. 3, 2025

(30) Foreign Application Priority Data
Mar. 30, 2022 (KR) 10-2022-0039463

(51) Int. Cl. G16H 50/20; G06N 3/08; G16H 10/60; G16H 50/50

(52) U.S. Cl. CPC G16H 50/20; G06N 3/08; G16H 10/60; G16H 50/50

(57) ABSTRACT

A deep neural network-based disease-of-interest prediction device according to the present disclosure may include a data collection unit that collects patient-specific medical diagnosis data, an input data generation unit that generates input data by embedding each patient's medical diagnosis data into a binary vector, a disease-of-interest prediction model generation unit that learns the input data as learning data to generate a deep neural network-based disease-of-interest prediction model, the disease-of-interest prediction model generation unit generating compressed data with a reduced dimension of the input data and learning, when the compressed data is input, to output correct answer data corresponding to the compressed data so as to generate the disease-of-interest prediction model, and a disease-of-interest prediction unit that inputs the input data into the disease-of-interest prediction model to predict, according to the medical diagnosis data of the patient, whether a disease of interest has developed.

### TECHNICAL FIELD

The present disclosure relates to a device and method for predicting a disease of interest on the basis of a deep neural network, and a computer-readable program therefor, and more particularly, to a deep neural network-based disease-of-interest prediction device, method of predicting whether a patient has developed a disease of interest through a deep neural network-based disease-of-interest prediction model, and a computer-readable program therefor.

### BACKGROUND ART

Deep learning technology is a type of machine learning-based analysis that uses a layered algorithm architecture, and due to recent advances in deep learning technology, the deep learning technology is being applied to various fields including speech recognition, natural language processing, computer vision, and recommendation systems.

In addition, even in the medical field, the deep learning technology is being applied to medical image processing and analysis, natural language processing of large-scale medical text data, precision medicine, clinical decision support, and predictive analysis.

Meanwhile, in order to diagnose a patient's disease, methods such as direct diagnosis by a doctor or prediction through various health checkups are still applied. In particular, the incidence rate of stomach cancer in Korea is much higher than in other countries, so many domestic patients are still not free from the risk of developing stomach cancer. To this end, the incidence rate of stomach cancer has decreased by adopting a national methodology for early detection and treatment of stomach cancer through health checkups, but it still accounts for a large proportion compared to other countries. In the case of stomach cancer, even if it is discovered through direct diagnosis, the prognosis is not only poor because the cancer is often already in an advanced state, but also there is a time and financial burden associated with health checkups.

Therefore, prior to diagnosis through such direct health checkups, a technology is needed to predict whether a disease of interest has developed by using correlations with various diseases of interest as well as stomach cancer based on the patient's existing disease diagnosis history.

### CITATION LIST

### Patent Literature

(Patent Document 1) Korean Patent Registration No. 10-2053295

### DISCLOSURE OF INVENTION

### Technical Problem

According to one aspect of the present disclosure, it aims to provide a disease-of-interest prediction device and method of generating input data by embedding patient-specific medical diagnosis data into a binary vector, learning the generated input data to generate a disease-of-interest prediction model, and predicting whether a disease-of-interest has developed based on a patient's medical diagnosis data based thereon, and a computer-readable program therefor.

### Technical Solution

A disease-of-interest prediction device according to an embodiment of the present disclosure may include a data collection unit that collects patient-specific medical diagnosis data, an input data generation unit that generates input data by embedding each patient's medical diagnosis data into a binary vector, a disease-of-interest prediction model generation unit that learns the input data as learning data to generate a deep neural network-based disease-of-interest prediction model, the disease-of-interest prediction model generation unit generating compressed data with a reduced dimension of the input data and learning, when the compressed data is input, to output correct answer data corresponding to the compressed data so as to generate the disease-of-interest prediction model, and a disease-of-interest prediction unit that inputs the input data into the disease-of-interest prediction model to predict, according to the medical diagnosis data of the patient, whether a disease of interest has developed.

Meanwhile, the data collection unit may extract disease code information assigned to patients from the medical diagnosis data, wherein the disease code information is International Statistical Classification of Disease (ICD) codes assigned to patients.

Furthermore, the input data generation unit may count the types of ICD codes assigned to respective patients, and generate input data for each patient as a binary vector having a size corresponding to the counted types of the ICD codes, wherein the input data determines a binary value of the binary vector based on whether there is a diagnosis history for each ICD code for each individual patient.

Furthermore, the disease-of-interest prediction model may include an autoencoder that inputs the input data to generate the compressed data, and reconstructs the input data again based on the compressed data, a classification layer that predicts whether a disease of interest has developed based on the compressed data, and a cost function application unit that applies a cost function to calculate a reconstruction error of the autoencoder and a prediction error of the classification layer.

Furthermore, the autoencoder may include an encoder that maps the input data into a latent space dimension to output the compressed data toward a bottleneck layer, and a decoder that reconstructs the compressed data of the bottleneck layer into the input data, wherein the classification layer is configured with a multi-layer perceptron structure connected to the bottleneck layer to predict whether the disease of interest has developed through supervised learning that inputs compressed data of the bottleneck layer and outputs the correct answer data.

Furthermore, the cost function application unit may apply a final cost function as a linear sum of a first cost function that calculates a reconstruction error of the autoencoder and a second cost function that calculates a prediction error of the classification layer, and apply individual weights to the first cost function and the second cost function to apply the final cost function, wherein the disease-of-interest prediction model generation unit optimizes the autoencoder and classification layer to minimize a final cost value calculated as a result of applying the final cost function to generate the disease-of-interest prediction model.

A disease-of-interest prediction method according to another embodiment of the present disclosure, which is a deep neural network-based disease-of-interest prediction method performed in a disease-of-interest prediction device, may include collecting patient-specific medical diagnosis data, generating input data by embedding each patient's medical diagnosis data into a binary vector, learning the input data as learning data to generate a deep neural network-based disease-of-interest prediction model, and more specifically, generating compressed data with a reduced dimension of the input data and learning, when the compressed data is input, to output correct answer data corresponding to the compressed data so as to generate the disease-of-interest prediction model, and inputting the input data into the disease-of-interest prediction model to predict the development of the disease of interest based on the medical diagnosis data of the patient.

Meanwhile, the collecting of the patient-specific medical diagnosis data may include extracting disease code information assigned to patients from the medical diagnosis data, wherein the disease code information is International Statistical Classification of Disease (ICD) codes assigned to patients.

Furthermore, the generating of the input data may include counting the types of ICD codes assigned to respective patients, and generating input data for each patient as a binary vector having a size corresponding to the counted types of the ICD codes, wherein the input data determines a binary value of the binary vector based on whether there is a diagnosis history for each ICD code for each individual patient.

Furthermore, the disease-of-interest prediction model may include an autoencoder that inputs the input data to generate the compressed data, and reconstructs the input data again based on the compressed data, a classification layer that predicts whether a disease of interest has developed based on the compressed data, and a cost function application unit that applies a cost function to calculate a reconstruction error of the autoencoder and a prediction error of the classification layer.

Furthermore, still another embodiment of the present disclosure may include a computer-readable program stored on a computer-readable recording medium, and the computer-readable program is configured to execute a deep neural network-based disease-of-interest prediction method.

### Advantageous Effects

According to the foregoing present disclosure, a disease of interest may be effectively predicted by using only a patient's existing diagnosis history.

Accordingly, there is an advantage capable of providing a patient with an opportunity for self-diagnosis, and allowing a doctor to efficiently perform predictive diagnosis of a disease of interest.

As a result, doctors may predict, without having to examining patients, whether the patient has a disease of interest using only his or her diagnosis history and perform additional examinations based on the prediction, thereby improving efficiency in the medical field.

### BRIEF DESCRIPTION OF THE DRAWINGS

FIG. 1 is a block diagram showing a configuration of a disease-of-interest prediction device according to an embodiment of the present disclosure.

FIG. 2 is a diagram showing a specific configuration of a disease-of-interest prediction model of a disease-of-interest prediction unit shown in FIG. 1.

FIG. 3 is a diagram showing each layer of a deep neural network that constitutes the disease-of-interest prediction model shown in FIG. 2.

FIG. 4 is a graph in which a disease-of-interest prediction performance according to the disease-of-interest prediction model shown in FIG. 2 is compared with those of other models.

FIG. 5 is a flowchart showing a disease-of-interest prediction method according to another embodiment of the present disclosure.

### BEST MODE FOR CARRYING OUT THE INVENTION

The detailed description of the present disclosure described below refers to the accompanying drawings, which show, by way of illustration, specific embodiments to carry out the present disclosure. These embodiments are described in sufficient detail to enable those skilled in the art to carry out the present disclosure. It should be understood that various embodiments of the present disclosure are different from one another but are not necessarily mutually exclusive. For example, specific shapes, structures and characteristics described herein may be implemented in other embodiments without departing from the concept and scope of the present disclosure in connection with one embodiment. In addition, it should be understood that the locations or arrangement of individual elements within each disclosed embodiment may be changed without departing from the concept and scope of the present disclosure. Therefore, the following detailed description is not to be taken in a limiting sense, and the scope of the present disclosure is defined only by the appended claims along with the entire scope of equivalents thereof, if properly described. The similar reference numerals refer to the same or similar functions in various aspects.

Hereinafter, preferred embodiments of the present disclosure will be described in more detail with reference to the drawings.

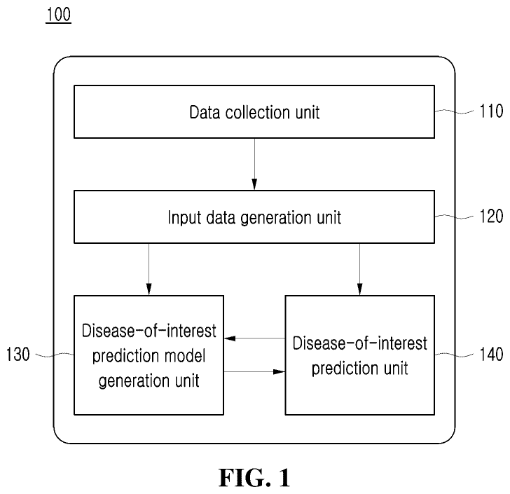

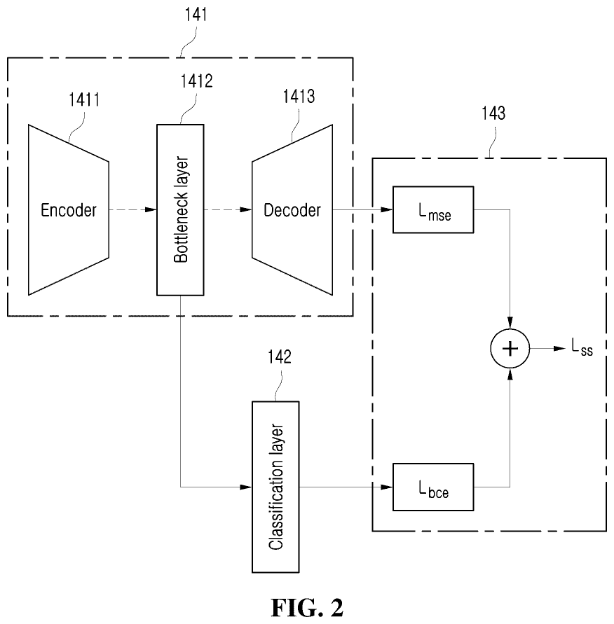

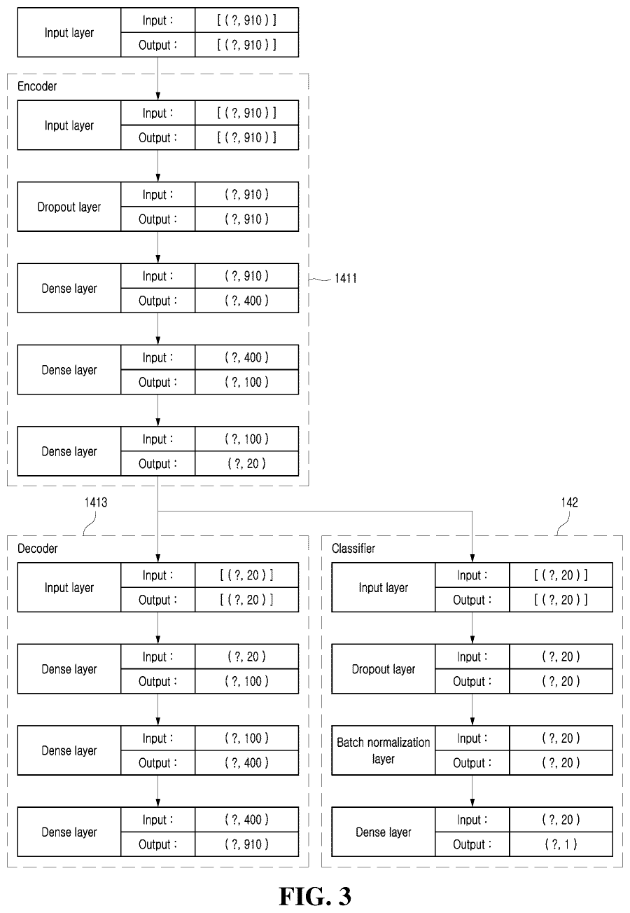

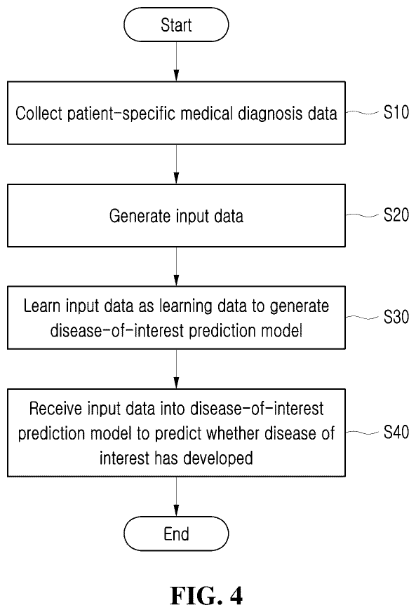

FIG. 1 is a block diagram showing a configuration of a disease-of-interest prediction device according to an embodiment of the present disclosure, FIG. 2 is a diagram showing a specific configuration of a disease-of-interest prediction model of a disease-of-interest prediction unit shown in FIG. 1, FIG. 3 is a diagram showing each layer of a deep neural network that constitutes the disease-of-interest prediction model shown in FIG. 2, and FIG. 4 is a graph in which a disease-of-interest prediction performance according to the disease-of-interest prediction model shown in FIG. 2 is compared with those of other models.

A disease-of-interest prediction device 100 according to this embodiment includes a data collection unit 110, an input data generation unit 120, a disease-of-interest prediction model generation unit 130, and a disease-of-interest prediction unit 140.

The data collection unit 110 collects patient-specific medical diagnosis data. To this end, the data collection unit 110 may collect medical diagnosis data by connecting with an external server, and store the collected medical diagnosis data linked to an ID assigned to each patient.

For example, the data collection unit 110 may collect medical diagnosis data provided by the National Health Insurance Corporation, and preferably, may randomly extract about 1 million patients and collect patient-specific medical diagnosis data for a specific year to secure training data for training a disease-of-interest prediction model.

The medical diagnosis data, which is demographic profiles and diagnostic data on diseases and related health problems, may include disease code information assigned to patients upon diagnosis by doctors.

Therefore, the data collection unit may extract disease code information assigned to patients from the medical diagnosis data. In this case, the disease code information may be International Statistical Classification of Disease (ICD) codes assigned to patients. Here, the ICD codes may include codes for diseases, signs and symptoms, abnormal findings, complaints, social situations, and external causes of injuries or diseases, and may be ICD codes based on the Korean Standard Classification of Diseases (KCD) as shown in Table 1 below.

TABLE 1

| Column Name | Description |
|---|---|
| IDV_ID | Unique patient ID |
| KEY_SEQ | Unique ID for each diagnosis |
| SEX | Gender of the subject |
| AGE_GROUP | Age-group (5 year-window) of the subject |
| DSBJT_CD | Medical department information |
| . . . | . . . |
| MAIN_SICK | Disease classification code (main) |
| SUB_SICK | Disease classification code (other than main) |
| . . . | . . . |
| RECU_FR_DT | Date of patient's visit |

The data collection unit 110 may extract only the primary or secondary ICD codes from the ICD codes. As a result, this ensures that only objective data is applied to a disease-of-interest prediction model, providing an advantage of training and predicting the disease-of-interest prediction model.

The input data generation unit 120 converts the medical diagnosis data collected from the data collection unit 110 into input data for application to the disease-of-interest prediction model. The input data may be used as learning data for training a disease-of-interest prediction model, or may be input into a trained disease-of-interest prediction model to be used as prediction data for predicting the disease of interest of the patient.

The input data generation unit 120 generates input data by embedding each patient's medical diagnosis data into a binary vector.

More specifically, the input data generation unit 120 first identifies the types of ICD codes assigned to respective patients, and counts the number of types of ICD codes found.

Furthermore, the input data generation unit 120 sorts ICD codes by patient ID. During this process, among the ICD codes, all ICD codes included in MAIN_SICK and SUB_SICK may be considered equal and sorted accordingly, with any duplicate ICD codes being removed.

Meanwhile, in order to increase the training efficiency of the disease-of-interest prediction model, the input data generation unit 120 may check respective ICD codes found in at least 50 different patients, select only patients with at least 6 different ICD codes from among the checked ICD codes, and sort those ICD codes to generate input data. For example, the input data generation unit may generate input data based on 910 ICD codes found in 712,050 patients.

The input data generation unit 120 may generate each patient-specific input data based on the sorted ICD codes. The input data generated in this manner may have the form of a binary vector, and the binary vector may have a size corresponding to the number of types of the counted ICD codes. Additionally, the binary value of the binary vector may be determined based on a result of sorting respective patient-specific ICD codes, that is, whether there is a diagnosis history for each ICD code. More specifically, the input data generation unit 120 may encode each ICD code to have a value of ‘1’ if there is a diagnosis history for an individual patient, and encode it to have a value of ‘0’ if there is no diagnosis history, thereby generating a binary vector having a binary value of ‘1’ or ‘0’ for each patient according to the diagnosis history.

Meanwhile, the input data generation unit 120 may generate input data in the form of a binary multi-matrix in which rows and columns represent patients and ICD codes, respectively. For example, a matrix M (i;j)(0,1) may represent a diagnostic history of patient i for ICD code j.

The disease-of-interest prediction model generation unit 130 learns input data as learning data to generate a disease-of-interest prediction model based on a deep neural network.

To this end, the disease-of-interest prediction model generation unit 130 generates compressed data with a reduced dimension of input data, and learns, when the compressed data is input, to output correct answer data corresponding to the compressed data to generate the disease-of-interest prediction model.

The disease-of-interest prediction model generated by the disease-of-interest prediction model generation unit 130, which is a model that automatically discovers disease codes related to a disease of interest by using input data generated based on the diagnosis history of each patient's ICD code as input, includes an autoencoder 141, a classification layer 142, and a cost function application unit 143, as shown in FIG. 2.

First, the autoencoder 141, which is one of deep neural network models that learn the compression of input data in an unsupervised manner, may include an encoder 1411, and a decoder 1413. The autoencoder 141 is optimized to provide input data to the encoder 1411 and compare the input data with an output of the decoder 1413 to output original input data.

The encoder 1411, which is provided to learn how to compress input data into compressed data, maps the input data into a latent space dimension to output the compressed data toward a bottleneck layer 1412. A process of mapping the input data (x) to low-dimensional compressed data (z) of the bottleneck layer follows Equation 1 below.

$$z = {f}_{θ} ( x ) = {s}_{f} ( {W}_{e} x + {b}_{e} ) ∈ {R}^{{d}_{z}} \qquad [ Equation 1 ]$$

Here, Wₑ is a weight matrix, bₑ is a bias, and sⱼ ( ) is an activation function.

The decoder 1413, which is provided to learn a method of reconstructing compressed data into original input data, reconstructs compressed data from the bottleneck layer 1412 into input data to output the reconfigured data. A process of reconstructing the compressed data (z) into original input data to output the reconfigured data (y) follows Equation 2 below.

$$y = {g}_{ϕ} ( z ) = {s}_{g} ( {W}_{d} z + {b}_{d} ) \qquad [ Equation 2 ]$$

Here, Wₐ and bₐ are parameters of a decoder, and s_{g} ( ) is an activation function.

Meanwhile, the encoder 1411 and decoder 1413 according to this embodiment may be configured with a plurality layers, as shown in FIG. 3. More specifically, the encoder 1411 may be configured with an input layer (InputLayer), a dropout layer (Dropout), and a plurality of dense layers (Dense), and the decoder 1413 may be configured with an input layer and a plurality dense layers (Dense). Accordingly, in an encoder and decoder having a single layer structure, an error may be further reduced to improve compression performance.

The classification layer 142 is connected to the bottleneck layer 1412 of the autoencoder 141 to predict whether a disease of interest has developed based on compressed data. That is, the classification layer 142 according to this embodiment does not receive input data in the form of a binary multi-matrix itself, but receives compressed data with a reduced dimension to predict whether a disease of interest has developed.

The classification layer 142 may be trained in a supervised learning manner by the disease-of-interest prediction model generation unit 130, and the disease-of-interest prediction model generation unit 130 may input compressed data into the classification layer 142 and optimize the classification layer 142 to output correct answer data corresponding to the compressed data. In this case, the correct answer data may be a label value corresponding to the input data.

To this end, the classification layer 142 may be configured with a multi-layer perceptron structure having one output neuron, and more specifically, may be configured with a plurality of layers having an input layer (InputLayer), a dropout layer (Dropout), a batch normalization layer (BatchNormalization), and a dense layer (Dense), as shown in FIG. 3.

The cost function application unit 143 applies a cost function to calculate a reconstruction error of the autoencoder 141 and a prediction error of the classification layer 142.

To this end, the cost function application unit 143 may apply a final cost function Lₛₛ, which is a linear sum of a first cost function Lₘₛₑ and a second cost function L_{bce}. In other words, the cost function application unit 143 may calculate a reconstruction error of the autoencoder 141 through the first cost function Lₘₛₑ, calculate a prediction error of the classification layer 142 through the second cost function L_{bce}, and calculate a final cost value as a linear sum thereof.

Here, a reconstruction error L calculated according to the first cost function Lₘₛₑ follows Equation 3 below.

$$L ( x , y ) = {‖ x - y ‖}^{2} \qquad [ Equation 3 ]$$

Here, x is input data of the autoencoder 141, and y is output data reconstructed from the input data of the autoencoder 141.

Meanwhile, a prediction error BCE calculated according to the second cost function L_{bce} may be defined as a binary cross entropy, as in Equation 4 below.

$$BCE ( x ) = - \frac{1}{N} {\sum }_{i = 1}^{N} {y}_{i} log ( h ( {x}_{i} ; θ ) ) + ( 1 - {y}_{i} ) log ( 1 - h ( {x}_{i} ; θ ) ) \qquad [ Equation 4 ]$$

Here, yᵢ is a label value, which can be expressed as ‘1’ if the disease of interest has developed and ‘0’ if it has not developed. In addition, h(xᵢ;θ) may be defined as a function that has sequentially passed through f_{θ}(x) in Equation 1 and g_{ϕ}(z) in Equation 2.

Meanwhile, the cost function application unit 143 may apply a final cost function Lₛₛ by applying individual weights to the first cost function Lₘₛₑ and the second cost function L_{bce}.

That is, the cost function application unit 143 adjusts an application ratio of the two cost functions to increase the accuracy of predicting the disease of interest.

Therefore, the disease-of-interest prediction model generation unit 130 may generate a disease-of-interest prediction model by optimizing the autoencoder 141 and the classification layer 142 to minimize a final cost value calculated as a result of applying the final cost function Lₛₛ. The disease-of-interest prediction model generation unit 130 may generate a disease-of-interest prediction model according to an End-to-End Supervised AE (EEsAE) method that simultaneously updates the autoencoder 141 and the classification layer 142.

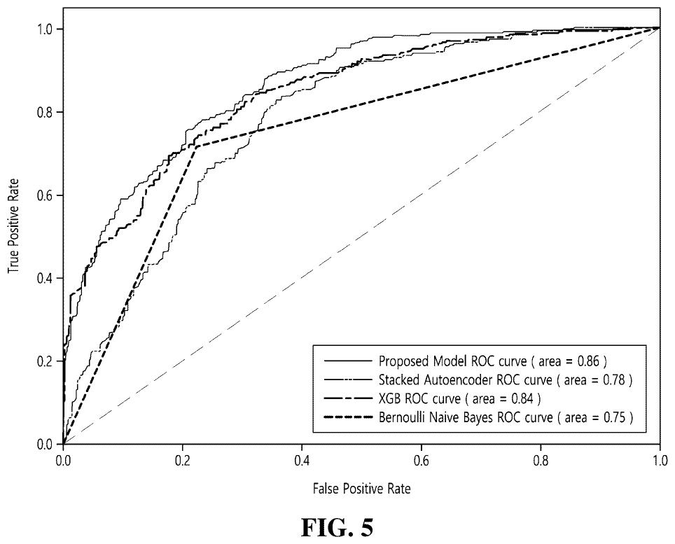

As shown in FIG. 5, it can be seen that the disease-of-interest prediction model generated in this manner shows a higher prediction performance than other models. FIG. 5 is a graph showing ROC curves, which are performance indicators of disease-of-interest prediction, when applying a disease-of-interest prediction model according to this embodiment and other reference models, such as a Stacked Autoencoder model, an Extreme Gradient Boosting (XGB) model, and a Naive Bayes model, and according to FIG. 5, an AUROC value of the disease-of-interest prediction model according to this embodiment is 0.86, and thus it can be seen that it exhibits a higher prediction performance than other reference models.

The disease-of-interest prediction unit 140 inputs the input data of a patient whose development of a disease of interest is to be checked into a disease-of-interest prediction model generated from the disease-of-interest prediction model generation unit 130 to predict whether the disease of interest has developed based on the medical diagnosis data of the patient. For example, the disease-of-interest prediction unit 140 may predict whether stomach cancer has developed based on the patient's medical diagnosis data, and is not limited thereto, and may also predict whether various diseases of interest have developed.

FIG. 5 is a flowchart showing a disease-of-interest prediction method according to another embodiment of the present disclosure.

A disease-of-interest prediction method according to this embodiment, which is a deep neural network-based disease-of-interest prediction method performed in a disease-of-interest prediction device, may include collecting patient-specific medical diagnosis data in a data collection unit (S10), generating input data by embedding each patient's medical diagnosis data into a binary vector in an input data generation unit (S20), learning the input data as learning data to generate a deep neural network-based disease-of-interest prediction model in a disease-of-interest prediction model generation unit (S30), and more specifically, generating compressed data with a reduced dimension of the input data and learning, when the compressed data is input, to output correct answer data corresponding to the compressed data so as to generate the disease-of-interest prediction model (S30); and inputting the input data into the disease-of-interest prediction model to predict, according to the medical diagnosis data of the patient, whether a disease of interest has developed (S40).

Meanwhile, the collecting of patient-specific medical diagnosis data (S10) may include extracting disease code information assigned to patients from the medical diagnosis data, and the disease code information may be International Statistical Classification of Disease (ICD) codes assigned to patients.

Furthermore, the generating of the input data (S20) may include counting the types of ICD codes assigned to respective patients and generating input data for each patient as a binary vector having a size corresponding to the counted types of the ICD codes, wherein the input data determines a binary value of the binary vector based on whether there is a diagnosis history for each ICD code for each individual patient.

In addition, the disease-of-interest prediction model may include an autoencoder that inputs the input data to generate the compressed data, and reconstructs the input data again based on the compressed data, a classification layer that predicts whether a disease of interest has developed based on the compressed data, and a cost function application unit that applies a cost function to calculate a reconstruction error of the autoencoder and a prediction error of the classification layer.

Other features are the same as those of the disease-of-interest prediction device described through FIGS. 1 to 3, and a description thereof will be omitted.

According to the foregoing present disclosure, a disease of interest may be effectively predicted by using only a patient's existing diagnosis history. Accordingly, there is an advantage capable of providing a patient with an opportunity for self-diagnosis, and allowing a doctor to efficiently perform predictive diagnosis of a disease of interest. As a result, doctors may predict, without having to examining patients, whether the patient has a disease of interest using only his or her diagnosis history and perform additional examinations based on the prediction, thereby improving efficiency in the medical field.

An operation by the disease-of-interest prediction method according to embodiments described above may be implemented, at least partially, as a computer program and recorded on a computer-readable recording medium. The computer-readable recording medium in which a program for implementing an operation by the disease-of-interest prediction method according to embodiments is recorded includes any type of recording device in which data that can be read by a computer is stored. Examples of the computer-readable recording medium include a ROM, a RAM, a CD-ROM, a magnetic tape, a floppy disk, an optical data storage device, and the like. Furthermore, the computer-readable recording medium may be distributed over computer systems connected via a network, and stored and executed as computer-readable codes in a distributed manner. In addition, functional programs, codes, and code segments for implementing this embodiment will be easily understood by those skilled in the art to which this embodiment belongs.

Though the present disclosure has been described above with reference to embodiments, it will be understood by those skilled in the art that various modifications and improvements can be made thereto without departing from the concept and scope of the present disclosure as set forth in the claims below.

### DESCRIPTION OF SYMBOLS

110: Data collection unit
120: Input data generation unit
130: Disease-of-interest prediction model generation unit
140: Disease-of-interest prediction unit

1. A deep neural network-based disease-of-interest prediction device, the device comprising:
a data collection unit that collects a medical diagnosis data of each patient;
an input data generation unit that generates input data by embedding the medical diagnosis data of each patient into a binary vector;
a disease-of-interest prediction model generation unit configured to learn the input data as learning data to generate a deep neural network-based disease-of-interest prediction model, the disease-of-interest prediction model generation unit generating compressed data with a reduced dimension of the input data and learning, when the compressed data is input, to output correct answer data corresponding to the compressed data so as to generate the disease-of-interest prediction model; and
a disease-of-interest prediction unit that inputs the input data into the disease-of-interest prediction model to predict, according to the medical diagnosis data of each patient, a development of disease of interest.

2. The device of claim 1, wherein the data collection unit extracts disease code information assigned to each patient from the medical diagnosis data, and
wherein the disease code information is International Statistical Classification of Disease (ICD) codes assigned to each patient.

3. The device of claim 2, wherein the input data generation unit counts types of the ICD codes assigned to each patient, and generates input data for each patient as a binary vector having a size corresponding to the types of the ICD codes, and
wherein the input data determines a binary value of the binary vector based on existence of a diagnosis history for each of the ICD codes for each patient.

4. The device of claim 1, wherein the disease-of-interest prediction model comprises:
an autoencoder configured to input the input data to generate the compressed data, and reconstruct the input data based on the compressed data;
a classification layer configured to predict the development of whether a disease of interest based on the compressed data; and
a cost function application unit configured to apply a cost function to calculate a reconstruction error of the autoencoder and a prediction error of the classification layer.

5. The device of claim 4, wherein the autoencoder comprises an encoder that maps the input data into a latent space dimension to output the compressed data toward a bottleneck layer, and a decoder configured to reconstruct the compressed data of the bottleneck layer into the input data, and
wherein the classification layer is configured with a multi-layer perceptron structure connected to the bottleneck layer to predict whether the disease of interest through supervised learning that inputs compressed data of the bottleneck layer and outputs the correct answer data.

6. The device of claim 5, wherein the cost function application unit applies a final cost function as a linear sum of a first cost function configured to calculate a reconstruction error of the autoencoder and a second cost function configured to calculate a prediction error of the classification layer, and apply individual weights to the first cost function and the second cost function to apply the final cost function, and
wherein the disease-of-interest prediction model generation unit configured to optimize the autoencoder and classification layer to minimize a final cost value calculated as a result of applying the final cost function and to generate the disease-of-interest prediction model.

7. A deep neural network-based disease-of-interest prediction method performed in a disease-of-interest prediction device, the method comprising:
collecting a medical diagnosis data of each patient;
generating input data by embedding the medical diagnosis data of each patient into a binary vector;
learning the input data as learning data to generate a deep neural network-based disease-of-interest prediction model,
wherein the learning the input data comprising generating compressed data with a reduced dimension of the input data and learning, when the compressed data is input, to output correct answer data corresponding to the compressed data and to generate the disease-of-interest prediction model; and
inputting the input data into the disease-of-interest prediction model to predict, according to the medical diagnosis data of each patient, a development of disease of interest.

8. The method of claim 7, wherein the collecting of the medical diagnosis data of each patient comprises:
extracting disease code information assigned to each patient from the medical diagnosis data, and
wherein the disease code information is International Statistical Classification of Disease (ICD) codes assigned to each patient.

9. The method of claim 8, wherein the generating of the input data comprises:
counting types of the ICD codes assigned to each patient; and
generating input data for each patient as a binary vector having a size corresponding to the types of the ICD codes, and
wherein the input data determines a binary value of the binary vector based on existence of a diagnosis history for each of the ICD codes for each patient.

10. The method of claim 7, wherein the disease-of-interest prediction model comprises:
an autoencoder configured to input the input data to generate the compressed data, and reconstruct the input data again based on the compressed data;
a classification layer configured to predict the development of the disease of interest based on the compressed data; and
a cost function application unit configured to apply a cost function to calculate a reconstruction error of the autoencoder and a prediction error of the classification layer.

11. A computer-readable program stored on a non-transitory computer-readable recording medium, wherein the computer-readable program configured to execute the deep neural network-based disease-of-interest prediction method of claim 7.
